(19) United States
(12) Patent Application Publication
(10) Pub. No.: US 2025/0213295 A1
(43) Pub. Date: Jul. 3, 2025

(54) ELECTROPORATION ABLATION FROM TISSUE-CONTACTING ELECTRODES

(71) Applicant: Boston Scientific Scimed, Inc., Maple Grove, MN (US)

(72) Inventors: Brendan Early Koop, Ham Lake, MN (US); Eric Daniel Buck, Shoreview, MN (US); Kara Elizabeth Garrott, Minneapolis, MN (US); Nathan Paul Hagstrom, Blaine, MN (US); Nathaniel Adams, New Richmond, WI (US)

(21) Appl. No.: 18/991,089

(22) Filed: Dec. 20, 2024

Related U.S. Application Data
(60) Provisional application No. 63/616,281, filed on Dec. 29, 2023.

(51) Int. Cl. A61B 18/14; A61B 17/00; A61B 18/00

(52) U.S. Cl. CPC A61B 18/1492; A61B 2017/00725; A61B 2018/00357; A61B 2018/00577; A61B 2018/00613; A61B 2018/00702; A61B 2018/00875; A61B 2018/1417

(57) ABSTRACT

A system to perform electroporation ablation of target tissue in a chamber of a patient's heart, the system comprising a catheter and a controller. The catheter includes an electrode assembly having a plurality of ablation electrodes, wherein the catheter is adapted to position the electrode assembly in contact with the target tissue. The controller is configured to identify, from among the plurality of ablation electrodes, a first subset of ablation electrodes that are in contact with the target tissue, and a second subset of ablation electrodes that are not in contact with the target tissue, and to apply a pulsed electrical signal to the first subset of ablation electrodes and not to the second subset of ablation electrodes.

### CROSS REFERENCE TO RELATED APPLICATIONS

This application claims priority to U.S. Provisional Patent Application No. 63/616,281, filed Dec. 29, 2023, which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a system to perform electroporation ablation of target tissue in a chamber of a patient's heart, the system comprising a catheter and a controller. The catheter includes an electrode assembly having a plurality of ablation electrodes, wherein the catheter is adapted to position the electrode assembly in contact with the target tissue. The controller is configured to identify, from among the plurality of ablation electrodes, a first subset of ablation electrodes that are in contact with the target tissue, and a second subset of ablation electrodes that are not in contact with the target tissue, and apply a pulsed electrical signal to the first subset of ablation electrodes and not to the second subset of ablation electrodes.

In Example 2, the system of Example 1, wherein the controller is configured to select one or more characteristics of the pulsed electrical signal based on a configuration of the first subset of ablation electrodes.

In Example 3, the system of Example 2, wherein the one or more characteristics of the pulsed electrical signal includes one or more waveform parameters selected based on the configuration of the first subset of the ablation electrodes.

In Example 4, the system of any of Examples 1-3, wherein the pulsed electrical signal includes a plurality of pulsed electrical signals generated in a pulse train, and wherein the controller is configured to identify the first subset of ablation electrodes before applying each pulsed electrical signal in the pulse train.

In Example 5, the system of any of Examples 1-4, further comprising an indifferent electrode configured to be operably coupled to the patient, and the controller is configured to apply the pulsed electrical signal to the first subset of ablation electrodes and the indifferent electrode configured in a monopolar mode.

In Example 6, the system of any of Examples 1-5, wherein the controller is configured to apply a pre-pulse electrical signal to the plurality of ablation electrodes and to identify the first subset of ablation electrodes and the second subset of ablation electrodes based on the pre-pulse electrical signal.

In Example 7, the system of Example 6, wherein the controller is configured to apply the pre-pulse electrical signal to the plurality of ablation electrodes configured in a bipolar mode.

In Example 8, the system of either of Examples 6 and 7, wherein the pre-pulse electrical signal is one of a continuous signal or a selectively applied, discrete pre-pulse signal.

In Example 9, the system of Example 8, wherein the pre-pulse signal is a selectively applied, discrete pre-pulse signal, and the controller is configured to apply a first pre-pulse signal with the electrode assembly at a first location known to be not in contact with the target tissue and a second pre-pulse signal with the electrode assembly at a second location in which at least one of the ablation electrodes is known to be in contact with the target tissue.

In Example 10, the system of Example 9, wherein the first pre-pulse signal is applied with the catheter disposed in a blood pool.

In Example 11, the system of Examples 1-5, wherein the electrode assembly further includes a plurality of sense electrodes.

In Example 12, the system of Example 11, wherein the controller is configured to apply a pre-pulse electrical signal to the plurality of ablation electrodes and the plurality of sense electrodes, and to identify the first subset of ablation electrodes and the second subset of ablation electrodes based on the pre-pulse signal.

In Example 13, the system of any of Examples 6-12, wherein the controller is configured to measure tissue impedance in the target tissue in response to the application of the pre-pulse signal and to identify the first subset of electrodes and the second subset of ablation electrodes based on the measured tissue impedance.

In Example 14, the system of any of Examples 1-3, wherein the controller is configured to be operable to perform a plurality of tissue-contact assessment processes, and the controller is further configured to select a tissue-contact assessment process for identifying the first subset of ablation electrodes and the second subset of ablation electrodes.

In Example 15, the system of Example 14, wherein the plurality of tissue-contact assessment processes include determining a degree of deformation of the electrode assembly due to a force applied by the target tissue.

In Example 16, a system to perform electroporation ablation of target tissue in a chamber of a patient's heart, the system comprising a catheter and a controller. The catheter includes an electrode assembly having a plurality of ablation electrodes, wherein the catheter is adapted to position the electrode assembly in contact with the target tissue. The controller is configured to determine a first subset of ablation electrodes in contact with tissue and a second subset of ablation electrodes that are not in contact with the target tissue, and selectively apply a pulsed electrical signal to only the first subset of ablation electrodes.

In Example 17, the system of Example 16, wherein the controller is configured to generate and apply a pre-pulse electrical signal to the plurality of ablation electrodes to determine the first subset of ablation electrodes and the second subset of ablation electrodes.

In Example 18, the system of Example 17, wherein the pre-pulse electrical signal is one of a continuous signal and a selectively applied, discrete pre-pulse signal.

In Example 19, the system of Example 16, wherein the controller is configured to apply a first pre-pulse signal to the plurality of ablation electrodes with the catheter not in contact with the target tissue and a second pre-pulse signal to the plurality of ablation electrodes with the catheter in contact with the target tissue.

In Example 20, the system of Example 19, wherein the controller is configured to apply the first pre-pulse signal with the electrode assembly disposed in a blood pool.

In Example 21, the system of Example 17, wherein the electrode assembly further includes a plurality of sense electrodes.

In Example 22, the system of Example 21, wherein the controller is further configured to generate and apply the pre-pulse electrical signal to the plurality of sense electrodes in addition to the plurality of ablation electrodes to determine the first subset of ablation electrodes and the second subset of ablation electrodes.

In Example 23, the system of Example 17, wherein the controller is configured to measure a target tissue impedance responsive to the application of the pre-pulse signal to the plurality of ablation electrodes.

In Example 24, the system of Example 16, wherein the controller is configured to select one or more characteristics of the pulsed electrical signal based on a configuration of the first subset of ablation electrodes.

In Example 25, the system of Example 24, wherein the one or more characteristics of the pulsed electrical signal includes one or more waveform parameters.

In Example 26, a system to perform electroporation ablation of target tissue in a chamber of a patient's heart, the system comprising a catheter and a controller. The catheter includes an electrode assembly having a plurality of ablation electrodes, wherein the catheter is adapted to position the electrode assembly in contact with the target tissue. The controller is configured to calibrate the plurality of ablation electrodes when the plurality of ablation electrodes are at a first location not in contact with the target tissue, determine, with the plurality of ablation electrodes at a second location, a subset of the plurality of ablation electrodes that are in contact with the target tissue based on the calibration, and selectively apply a pulsed electrical signal only to the subset of ablation electrodes.

In Example 27, the system of Example 26, wherein the controller is configured to apply a first pre-pulse signal to the plurality of ablation electrodes at the first location to calibrate the plurality of ablation electrodes, and to apply a second pre-pulse signal to the plurality of ablation electrodes at the second location to determine the subset of ablation electrodes in contact with the target tissue.

In Example 28, a method for use with a catheter including an electrode assembly having a plurality of ablation electrodes, wherein the catheter is adapted to position the electrode assembly in contact with the target tissue, the method comprising determining a subset of the plurality of ablation electrodes that are in contact with the target tissue, and selectively applying a pulsed electrical signal only to the subset of ablation electrodes.

In Example 29, the method of Example 28, further comprising selecting one or more characteristics of the pulsed electrical signal based on a configuration of the subset of ablation electrodes.

In Example 30, the method of Example 28, wherein determining the subset of ablation electrodes in contact with the target tissues includes applying a pre-pulse electrical signal to the plurality of ablation electrodes.

In Example 31, the method of Example 30, further comprising:
positioning the electrode assembly at a first location not in contact with the target tissue and applying the pre-pulse signal to the plurality of ablation electrodes at the first location;
establishing a first signal response measurement responsive to applying the pre-pulse signal to the plurality of ablation electrodes at the first location;
positioning the electrode assembly at a second location and applying the pre-pulse signal to the plurality of ablation electrodes at the second location; and
determining a second signal response measurement responsive to applying the pre-pulse signal to the plurality of ablation electrodes at the second location,
wherein determining the subset of ablation electrodes in contact with the target tissue is based on the first signal response measurement and the second signal response measurement.

In Example 32, the method of Example 31, wherein the first signal response measurement and the second signal response measurement each comprises an impedance responsive to the pre-pulse signal.

In Example 33, the method of Example 30, wherein the pre-pulse signal is a continuous signal or a selectively applied, discrete pre-pulse signal.

In Example 34, the method of Example 30, wherein the applying the pre-pulse electrical signal to the plurality of ablation electrodes includes applying the pre-pulse signal to the plurality of ablation electrodes with two or more of the plurality of electrodes configured in a bipolar mode.

In Example 35, the method of Example 34, wherein the applying the pulsed electrical signal to the plurality of ablation electrodes includes applying the pulsed electrical signal to one or more of the plurality of ablation electrodes configured in a monopolar mode in combination with an indifferent electrode.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIGS. 2A and 2B are perspective and end view illustrations, respectively, of a distal portion of a splined catheter for use in the electrophysiology system of FIG. 1, in accordance with embodiments of the subject matter of the disclosure.

FIG. 3 is a block diagram illustrating an example controller for use with the example electrophysiology system of FIG. 1.

FIG. 4 is a block diagram illustrating an example configuration of the example controller of FIG. 3.

FIG. 5 is a block diagram illustrating an exemplary process for configuring the controller of FIG. 5.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator, to generate an electric field of sufficient strength to create transmural lesions. In one example, one or more catheters may be advanced in a minimally invasive fashion through vasculature to a target location, such as in the heart. The methods described here may include introducing a device into an endocardial space of the heart. A pulse waveform may be generated and delivered to electrodes of the device to ablate tissue.

Some electroporation devices can include catheters having a three-dimensional electrode array, such ablation electrodes disposed on spline, baskets or balloons. In such a configuration, some ablation electrodes can be contact with the target tissue and other ablation electrodes are not in contact with the target tissue during the ablation procedure. For instance, ablation electrodes not in contact with the target tissue during ablation procedures can be disposed in a blood pool in a cardiac cavity. Activation of ablation electrodes not in contact with the target tissue during ablation can cause adverse effects among which can include bubble formation and skeletal muscle stimulation.

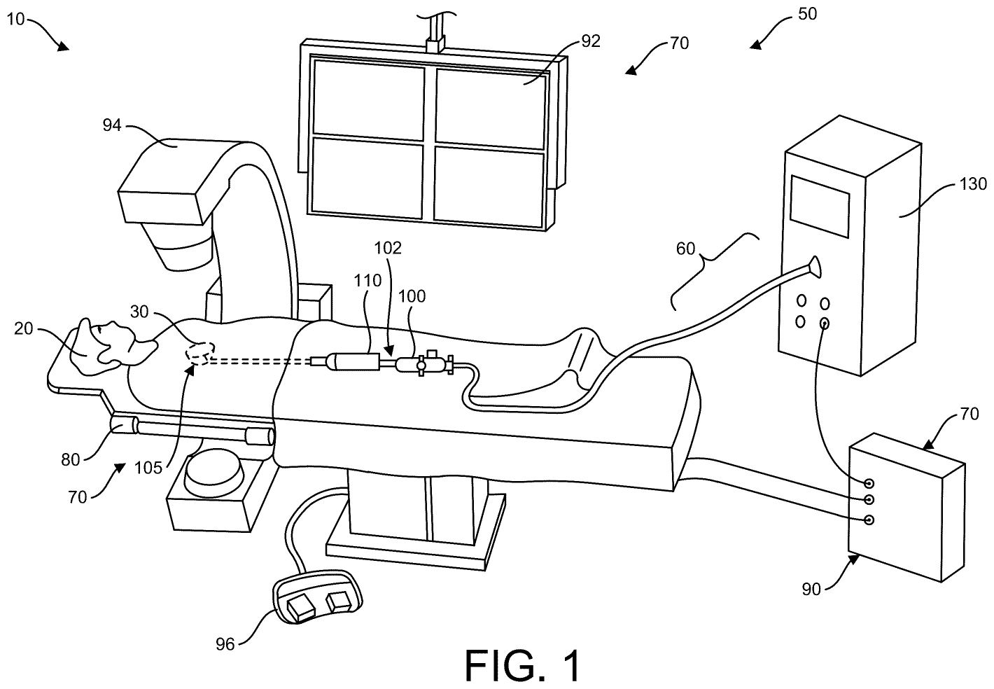

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables and tubing, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. The electroporation catheter system 60 can include additional features.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein often in the groin or possibly in the shoulder or neck. Once access to the vessel is obtained, the electroporation catheter 105 can be navigated to within the patient's heart, such as within a chamber of the heart.

In one example, the electroporation catheter 105 is a mapping and ablation catheter, which can be deployed in mapping procedures in cooperation with the EAM system 70 as well as to deliver ablation electric field energy and ablate tissue via irreversible electroporation. The example electroporation catheter 105 includes an elongated catheter shaft and distal end region configured to be deployed proximate target tissue, such as within a chamber of the patient's heart or the wall of a pulmonary vein ostium. The shaft can extend from an access point in the patient to the target tissue and generally defines a longitudinal axis of the electroporation catheter 105. A proximal end region of the catheter can include a handle having user manipulatable controls for the catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of flexible support members configured to form a basket, and at least a some of the electrodes are disposed on the flexible support members.

The electroporation catheter 105 is configurable in a plurality of states. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state. The electrode assembly has a collapsed shape when the catheter 105 is in the collapsed state and an expanded shape when the catheter 105 is in the expanded state. In some examples, the electrode assembly has more than two states.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

In the illustrated examples, the catheter 105 is a mapping and ablation catheter, and the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

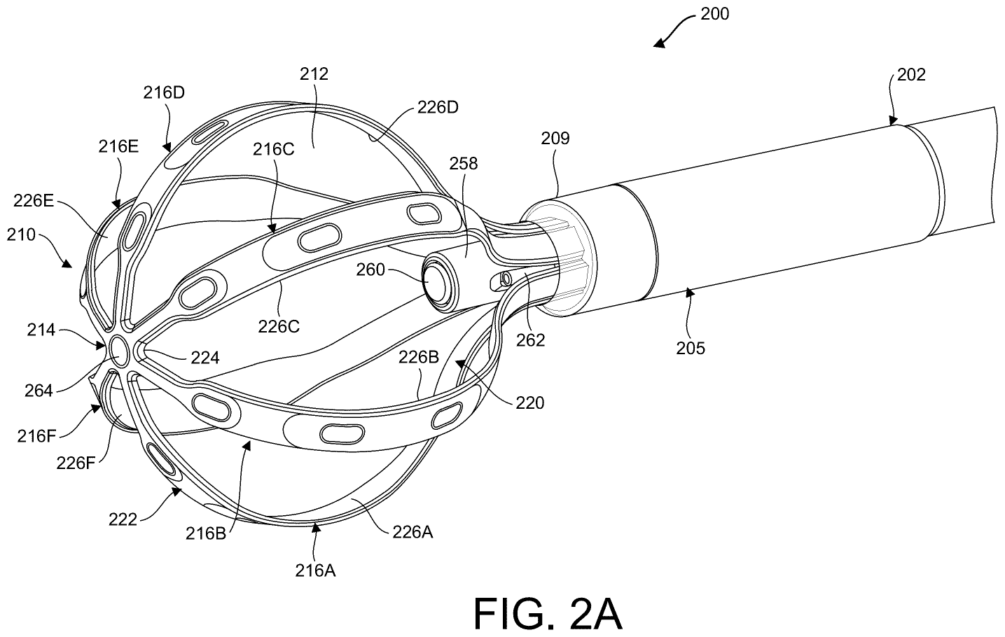

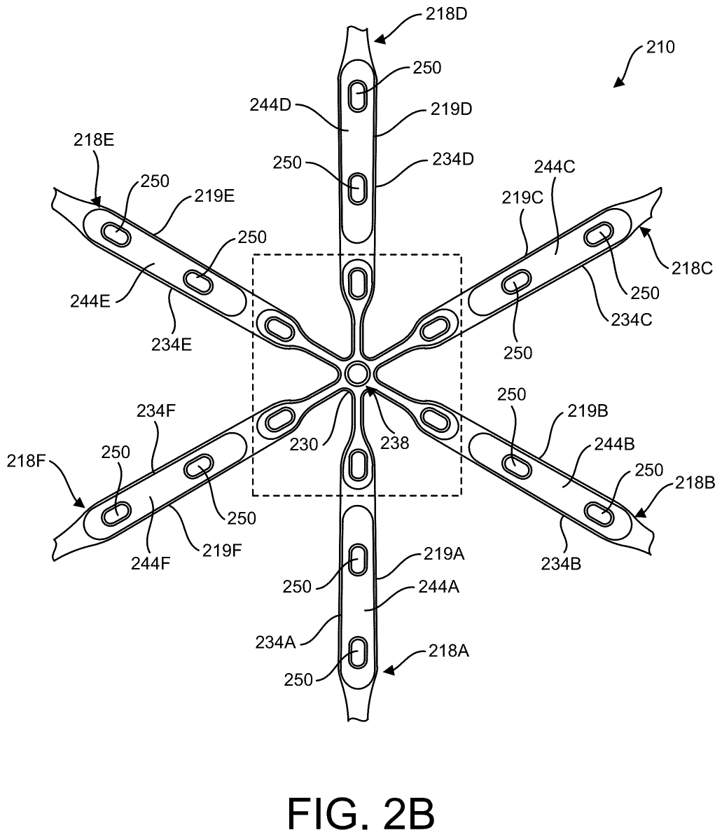

FIGS. 2A and 2B illustrate an example electroporation catheter 200 for mapping ablation of cardiac tissue, which can be an example of catheter 105 and used with the electrophysiology system 50. FIG. 2A is a partial perspective illustration of an electroporation catheter 200 having a catheter distal portion 205. The electroporation catheter 200 has a tubular outer shaft 208 having a shaft distal end 209, and an electrode assembly 210 extending distally from the distal end 209 of the outer shaft 208. In embodiments, the electrode assembly 210 is configured to self-expand from a collapsed state when constrained within a delivery sheath to a pre-defined expanded state defining an inner space, or cavity 212. The electrode assembly includes multiple ablation electrodes configured to receive pulsed electrical signals or waveforms from the electroporation console 130, for creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy such as via the EAM system 70, and determining proximity to target tissue within the anatomy.

FIG. 2B is a partial plan illustration of the electrode assembly 210 of the electroporation catheter 200 in two dimensions to demonstrate the layout of the electrode assembly 210. Referring to FIGS. 2A and 2B together, the electrode assembly 210 has a distally-located central hub portion 214 and a plurality of splines 216A-216F extending proximally from the central hub portion 214. Each respective spline 216A-216F has a distal end portion 217A-217F, a proximal end portion 218A-218F, and an intermediate portion 219A-219F extending between the distal end portion 217A-217F and the proximal end portion 218A-218F. Each of the proximal end portions 218A-218F is attached to and constrained by the distal end 209 of the outer shaft 202.

The splines 216A-216F include a support member 220 and a flexible circuit 222. The flexible circuit 222 is secured to and disposed over an outer surface of the support member 222. The support member 220 includes a support member hub 224 and a plurality of support member branches 226A-226F. The support member 220 provides a structural support of the electrode assembly 210. The support member 220 is formed from a superelastic material, such as a metal or polymer, to provide desired mechanical or structural properties to the electrode assembly 210. In one example, the support member 220 is formed from a superelastic metal alloy such as a nickel-titanium alloy. The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches 226A-226F.

The flexible circuit 222 includes a distal ablation electrode 238 that has a distal ablation electrode hub portion 240 and a plurality of radial segments 242A-242F. In the example, the distal ablation electrode hub portion 240 is located on the flex circuit hub 230. Additionally, the radial segments 242A-242F formed with the distal ablation electrode hub portion 240. Each of the radial segments 242A-242F extends proximally along a portion of a respective one of the flex circuit branches 234A-234F. The flexible circuit 222 further includes a plurality of proximal ablation electrodes 244A-244F. Each of the proximal ablation electrodes 244A-244F is located on a respective one of the flex circuit branches 234A-234F.

The flexible circuit 222 includes a plurality of spline sensing electrodes 250. In the example, each of the spline sensing electrodes 250 is disposed within a periphery of one of the proximal ablation electrodes 244A-244F or one of the radial segments 242A-242F of the distal ablation electrode 238. For instance, each of the distal-most spline sensing electrode 250 is disposed within a periphery of a respective one of the radial segments 242A-242F of the distal ablation electrode 238 and is electrically isolated from the distal ablation electrode 238. Additionally, a plurality of the more proximally-located spline sensing electrodes 250 is disposed along and within a periphery of a respective one of each of the proximal ablation electrodes 244A-244F and electrically isolated from the proximal ablation electrodes 244A-244F.

In the example, the electroporation catheter 200 includes a central post 258 extending distally from the distal end 209 of the outer shaft 202. The central post 258 extends partially into the cavity 212 and includes a post electrode 260. The post electrode 260 can operate as a reference for unipolar electrograms, in lieu of reliance on surface ECG patch electrodes. An irrigation lumen 261 can be included and supported by the central post 258. The central post 258 may house additional components such as a magnetic navigation sensor. In the illustrated embodiment, the electrode assembly 210 further includes a hub sensing electrode 264 centrally located on the flex circuit hub 230.

The post electrode 260 can provide additional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70.

The hub sensing electrode 264 allows tissue surface mapping to be conducted in a “forward” manner, reducing the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

The example electrode assembly 210 is primarily designed to create relatively localized ablation lesions, or focal lesions, as compared to relatively large diameter circumferential lesions that may be created in pulmonary vein isolation procedures. However, the present disclosure can be readily adapted for a catheter capable of large diameter circumferential lesions. The example electrode assembly 210 can provide a clinician with a wide range of capabilities for monopolar and bipolar focal pulsed field ablation of cardiac tissue, combined with the ability to perform localized, such as at the location of the delivery of pulsed field ablative energy, high fidelity sensing of cardiac tissue, for lesion or conduction block assessment, or tissue contact determinations.

The depiction of the electroporation catheter 200 is intended for illustration or a general overview of components of a catheter for use in system 50 and is not intended to imply that the disclosure is limited to any set of components or arrangement of the components. For example, another electroporation catheter, such as catheters with three-dimensional electrode arrays including basket catheters or ablation-only catheters, can be included in the electrophysiology system 50.

The example of the electroporation catheter 200 includes seven ablation electrodes and nineteen sensing electrodes that can be placed in contact with the target tissue in addition to other electrodes or sensors that are not expected to be placed in contact with the target tissue. The separate ablation electrodes are employed to produce appropriate lesion depths for the treatment of atrial arrythmias. Ablation procedures with the electroporation catheter 200 can be performed in a monopolar mode with an indifferent or patch electrode applied to a patient's skin, such as skin on the patient's back. Generally during an ablation procedure, some of the ablation electrodes in the electrode array are in contact with the target tissue while some of the ablation electrodes are not in contact with the target tissue. Rarely, if ever, are all ablation electrodes in contact with the target tissue in such a three-dimensional electrode array. The application of pulsed signals to ablation electrodes not in contact with the target tissue, such as ablation electrodes in the blood pool of the cardiac chamber, e.g., the left atrium, be undesirable for various clinical and/or operational reasons such as microbubble formation, skeletal muscle stimulation, reduced ablation effectiveness, and the like. The electrophysiology system 50 is employed to determine a subset of ablation electrodes in contact with the target tissue, wherein the subset of ablation electrodes is fewer than the plurality of ablation electrodes, and a remainder ablation electrode not in contact with the target tissue and apply a pulsed electrical signal to the subset of ablation electrodes and not to the remainder ablation electrode. In one example, the characteristics of the pulsed electrical signal is configured based on the subset of ablation electrodes.

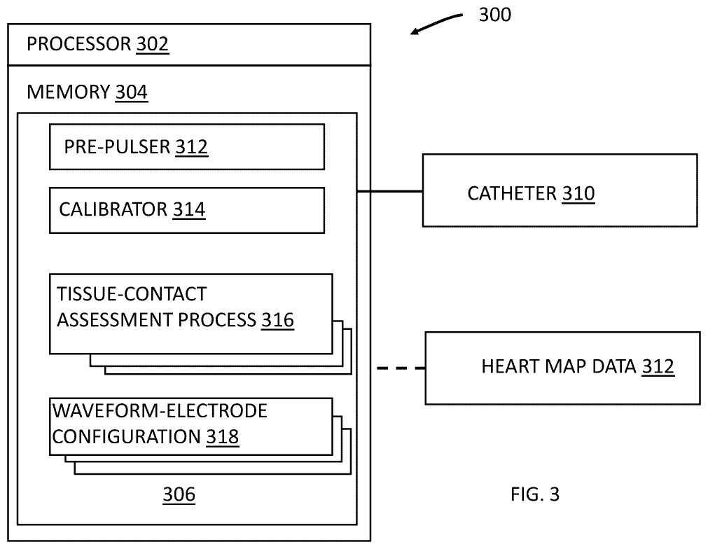

FIG. 3 illustrates an example controller 300 that can be used with the example electrophysiology system 50, such as a controller of the example catheter system 60 or the electroporation console 130. The controller 300 can be implemented to determine which of the ablation electrodes of an electroporation catheter, such as catheter 200, is in contact with target tissue and select or activate such ablation electrodes to provide PFA and deselect or deactivate ablation electrodes that are not in contact with target tissue. Further, the controller 300 can be implemented to adapt the PFA waveform provided to the selected ablation electrodes based on the configuration, e.g., the number, spacing, orientation etc. of the selected electrodes. The controller 300 can include a processor 302 and a memory 304. The memory 304 stores processor executable instructions 306. In one example, the processor executable instructions 306 can be in the form of a program, such as a computer program or application. The processor 302 can execute the instructions 306 that can be included in configuring the controller 300. In one example, the controller 300 can be implemented to include a computing device such as a laptop computer, a workstation, a desktop computer, a tablet, or a smartphone or devices such as the electroporation console 130 such as a PFA generator. In such examples, the controller 300 can include additional components such as a display, a touchscreen, speakers or other output devices, a keyboard or other input devices, or communication circuitry such as computer network adapters. The controller 300 may be implemented in a variety of architectures and components, such as the processor 302 and memory 304, may be distributed in various locations.

In one example, the processor 302 may include a plurality of main processing cores to run an operating system and perform general-purpose tasks on an integrated circuit. The processor 302 may also include built-in logic or a programmable functional unit, also on the same integrated circuit. In additional to multiple general-purpose, main processing cores and the application processing unit, controller 300 can include other devices or circuits such as graphics processing units or neural network processing units with the main processing cores. For example, the controller 300 may be used to perform other tasks.

Memory 304 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 302. Any such computer storage media may be part of the controller 300 and implemented as memory 304. Memory 304 is a non-transitory, processor readable memory device. Accordingly, a propagating signal by itself does not qualify as storage media or memory 304.

The controller 300 may be configured to receive inputs or information from the electrophysiology system 50, such as inputs from the electroporation catheter system 60 and EAM system 70 including the electroporation console 130 and the mapping and navigation controller 90, for storage in memory 304 and use by the instructions 306. For example, the controller receives inputs representative of data obtained with the catheter system 60, such as data determined from electrical signals received from the sensing electrodes and tracking devices, or catheter obtained data 308. Catheter obtained data 308 can include data determined from electrical signals of the heart, electrode location information, tissue proximity information, force or contact information, catheter tip or tissue temperature, acoustic information, catheter electrical coupling information, catheter deployment shape information, electrode properties, respiration phase, blood pressure, other physiological information. In some examples, catheter obtained data 308 can include or be supplemented with other information collected from sensors during mapping of the heart. In some examples, the controller 300 can receive an input representative of the anatomical map of the heart, or heart map data 310, which collected heart map data 310 can include the data regarding representations of the geometric anatomical map of the heart and the electro-anatomical map of the heart, such as from the EAM system 70. Heart map data 310 can include data previously collected, such as in the same procedure using a different map dataset, or with a different modality such as computerized tomography (CT), magnetic resonance imaging (MRI), ultrasound, or rotational angiography, and registered to the catheter locating system.

The program 306 can include a suite of modules stored in memory 304. For instance, the controller 300, via the program 306, can be configured to generate electrical signals with a pre-pulser 312 to provide to the catheter 105, and receive feedback from anatomy as part of catheter obtained data 308. The pre-pulser 312, via the electroporation console 130, can provide electrical signals to the ablation electrodes only and configured in a bipolar mode to determine impedance or current readings from feedback signals. In one example, the pre-pulser 312, via the electroporation console 130, can provide electrical signals to the ablation electrodes and the sensing electrodes configured in a bipolar mode to determine impedance or current readings from feedback signals. The pre-pulse electrical signal can be a continuous signal that is selectively read or can be a selectively applied, discrete pre-pulse signal that is read in response to being applied. The program 306 can includes a calibrator 314 to obtain a baseline measurement, such as readings when the catheter is not in contact with tissue that can be compared to readings when the catheter is in contact with tissue to determine which of the ablation electrodes are in contact with tissue. In one example, the calibrator 314 can be operative with the pre-pulser 312 to provide, for instance, a first pre-pulse signal with the catheter not in contact with the target tissue and a second pre-pulse signal with the catheter in contact with the target tissue, such as with a selectively applied, discrete pre-pulse signal. Additionally, the program can include one or more tissue contact assessment processes 316 that can be applied to determine which of the ablation electrodes are in contact with the target tissue. In the case of a plurality of tissue contact assessment processes 316, and the controller 300 is further configured to select a tissue contact assessment process from the plurality of tissue contact assessment processes. The memory 304 can also include an electrode-waveform map 318, which can be used to determine a corresponding pulsed electrical signal to be applied to the ablation electrodes in contact with the tissue based on the subset of the ablation electrodes in contact with the tissue. In one example, the electrode-waveform map 318 may be a lookup table, or a set of lookup tables stored in memory 304, although other configurations are contemplated. Still further, the program 306 can include an ablator 320 to apply the determine corresponding pulsed electrical signal to the subset of ablation electrodes to effect ablation by electroporation.

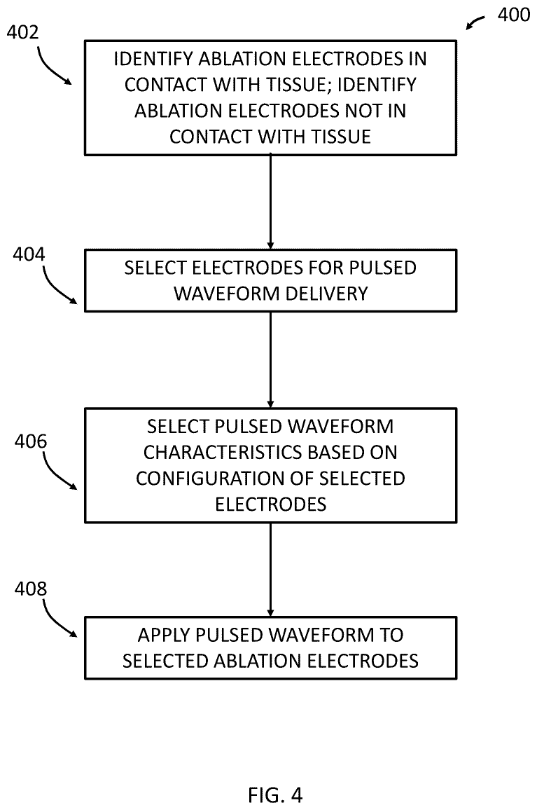

FIG. 4 illustrates a process 400 of configuring a controller, such as controller 300, to identify which of the ablation electrodes of an electroporation catheter, such as catheter 200, are in contact with target tissue when in use, and to select or activate such ablation electrodes to provide PFA and deselect or deactivate ablation electrodes that are not in contact with target tissue. Further, the controller 300 can be configured to adapt the PFA waveform provided to the selected ablation electrodes based on the configuration, e.g., number, spacing, orientation, etc. of the selected electrodes. For example, the controller can be implemented as part of the electroporation catheter system 60 or operably coupled to the EAM system 70. Process 400 can be use with a catheter including an electrode assembly having a plurality of ablation electrodes, wherein the catheter is adapted to position the electrode assembly in contact with the target tissue.

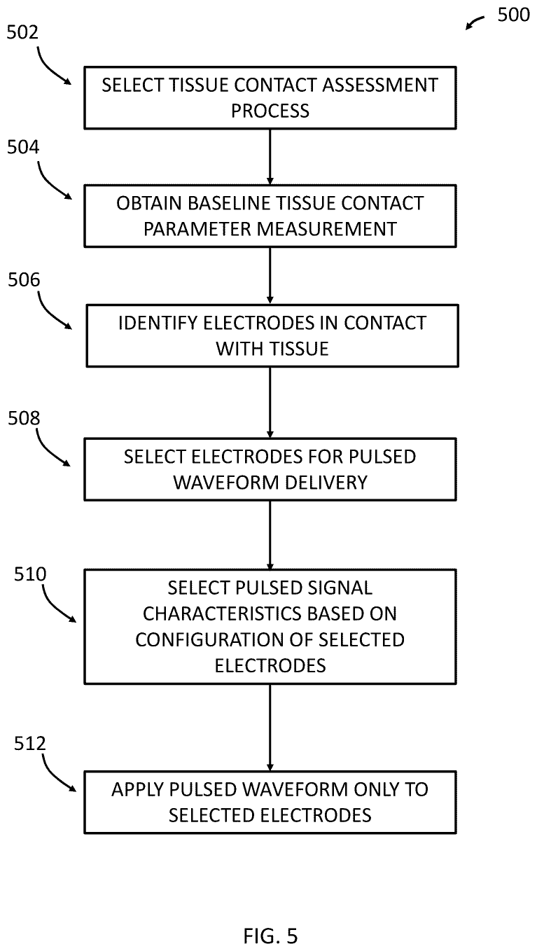

As shown, at 402, the controller can be configured to determine a subset of ablation electrodes in contact with the target tissue, wherein the subset of ablation electrodes is fewer than the plurality of ablation electrodes, and to determine a which of the ablation electrodes, if any, that are not in contact with the target tissue. Exemplary methodologies for identifying the tissue-contacting ablation electrodes are discussed in greater detail in connection with FIG. 5.

In the illustrated embodiment at 404, the controller can be configured to select ablation electrodes among the subset of ablation electrodes identified as being in contact with target tissue to which the pulsed waveform is to be delivered or applied. In some embodiments, all of the ablation electrodes identified as being in contact with tissue may be selected, or alternatively, fewer than all of the tissue-contacting electrodes may be selected for pulsed waveform delivery based on predetermined criteria.

At 406, the controller executes an algorithm to adapt a pulsed waveform by selecting one or more characteristics of the pulsed electrical signal are selected based on the configuration, e.g., the number, spacing, orientation etc. of the subset of ablation electrodes identified as being in contact with target tissue. For example, in embodiments, it may be clinically beneficial to spatially distribute the delivery of the pulsed waveform to selected electrodes as widely as possible to enhance the creation of wide-area focal ablation lesions. Accordingly, waveform characteristics such as the sequencing of pulsed waveform delivery to specific selected ablation electrodes (e.g., two or more spline ablation electrodes, or electrodes selected based on a desired active ablation electrode surface area) can be selected to optimize the tissue surface exposed to the ablative electric field created by the pulsed waveform. As an additional example, the overall duration of the pulsed waveform delivery, e.g., as in the number of heartbeats over which the pulsed waveform is delivered, can be dynamically selected by the controller based on the configuration of the selected ablation electrodes. In embodiments, one or more parameters of the pulsed electrical signal can also be adapted by the controller based on the selected ablation electrode configuration. Exemplary parameters may include, without limitation, pulse width, pulse spacing, pulse amplitudes (i.e., current, voltage), number of pulses per delivery, and the like. In still other embodiments, pulsed waveform parameters can be dynamically adjusted during delivery.

In embodiments, the selected ablation electrodes can be configured for delivery of ablative energy in either monopolar or bipolar modes. For example, in one embodiment, the selected ablation electrodes can be configured to operate in a monopolar mode, with an indifferent or return electrode located remotely from the catheter 105, e.g., as with a body-surface electrode. In embodiments, one or more of selected electrodes can be paired with one or more other ablation electrodes of the subset to form a bipolar ablation electrode pair for delivery of ablative PFA energy in a bipolar mode. In still other embodiments, a bipolar ablation electrode pair may be formed by one or more of the selected ablation electrodes and one or more other electrodes located on the catheter 105, e.g., one or more shaft electrodes located proximal to the electrode assembly.

As shown further shown in FIG. 4, at 408, the pulsed waveform is subsequently applied to only the selected ablation electrodes in accordance with the delivery strategy selected in step 406. In some embodiments, one or more pulsed waveform characteristics may additionally be dynamically adjusted by the controller after the initial delivery to the selected electrodes. For example, in an embodiment, an initial tissue impedance may be sensed by the ablation electrodes and/or sensing electrodes on the electrode assembly prior to delivery of the ablative pulse waveform, and subsequently adjusted during the ablative energy delivery, e.g., prior to each heartbeat or pulse packet delivery.

FIG. 5 illustrates a process 500 of configuring a controller, such as controller 300, to determine which of the ablation electrodes of an electroporation catheter, such as catheter 200, are in contact with target tissue, and to select or activate such ablation electrodes to provide PFA and deselect or deactivate ablation electrodes that are not in contact with target tissue. The controller 300 can be configured to adapt the PFA waveform provided to the selected ablation electrodes based on the configuration of the selected electrodes. For example, the controller can be implemented as part of the electroporation catheter system 60 or operably coupled to the EAM system 70. Process 500 can be an implementation or example of process 400. Process 500 can be use with a catheter including an electrode assembly having a plurality of ablation electrodes, wherein the catheter is adapted to be maneuvered in the chamber of the heart.

In the various embodiments, the system 50 may be configured to employ a range of tissue contact assessment processes. As shown in FIG. 5, at 502 one or more tissue contact assessment processes enabled by the system 50 (see FIG. 1) are selected, and at 504, a baseline measurement of one or more tissue contact parameters corresponding to the selected tissue contact assessment process(es) is made as a calibration step. Then at 506, after moving the electrode assembly to a location to which at least some portion of the electrode assembly is in contact with the target tissue, the controller 300 utilizes the selected tissue contact assessment to identify a subset of the ablation electrodes that are in contact with the target tissue, and then selects those ablation electrodes (or a subset of the tissue-contacting ablation electrodes) for delivery of the pulsed waveform at 508. Additionally, at 510, the controller 300 selects one or more pulsed signal characteristics for delivery to the selected ablation electrodes based on the configuration of the selected ablation electrodes, as described above in connection with process 400.

In one embodiment utilizing an impedance-based contact assessment process in which impedance between a given ablation electrode and a second electrode (e.g., another ablation electrode, a sensing electrode, or a remote indifferent electrode) is utilized as the tissue contact parameter, the controller is configured to deliver pre-pulses (which may be configured to generate electric fields below the threshold sufficient to ablate tissue) to the ablation electrodes and/or sense electrodes at the first location with the electrode assembly in the blood pool and then subsequently at a second location at which the electrode assembly is in contact with the target tissue, measure the responsive impedance at each ablation electrode at each location, and compare the measured impedances to assess which ablation electrodes at the second location are actually in contact with tissue and which remain in the blood pool (or which have only minimal contact with tissue).

In exemplary embodiments of the tissue contact assessment process, separate, discrete pre-pulses can be applied to the electrode assembly to determine differences between ablation electrodes and to select the electrodes with the greater current flow. In one example, the impedance or current measurement at an ablation electrode can be compared to the baseline measurement at the ablation electrode to determine whether the electrode is in contact with tissue. In another example, the impedance or current measurement at an ablation electrode can be compared to another ablation electrode in the electrode assembly, such as an ablation electrode opposite the electrode assembly, such as on an opposite or distant spline like ablation electrode 244A to ablation electrodes 244C or 244D. The pre-pulses can be provided in a bipolar mode, which may be more sensitive to local impedance determinations than in monopolar mode with an indifferent electrode applied as a patch. The bipolar mode can generate an electric field with the pre-pulse between two ablation electrodes or between an ablation electrode and a sense electrode. In another example, the pre-pulse can be a continuous electrical signal rather than a separate discrete signal, and changes in impedance or current flow can be tracked to determine ablation electrodes in contact with the target tissue. For example, phase shifts between voltage and current via impedance can be tracked to determine which of the ablation electrodes has come into contact with the target tissue. Again, ablation electrodes and sense electrodes can be employed with continuous pre-pulse signals.

Other processes can be employed to determine which of the ablation electrodes are in contact with the target tissue and which of the ablation electrodes are not in contact with the target tissue as well. For example, voltage measurements between electrodes can be taken as baseline measurement, and changes in voltage measurements can be used to determine deflection or shape changes in the electrode array, which can be used to determine which of the ablation electrodes are in contact with the target tissue and which of the ablation electrodes are not in contact with the target tissue. Force sensing can be employed with other sensors such as directional or navigational sensors or via rotation or bending of the shaft at the distal end. Electrogram sensing such as amplitude, slew rate, and other characteristics can be employed to confirm tissue contact. In some examples, an electrode array can include temperature sensors on the splines, and changes in temperature from a baseline measurement can be employed to determine which electrodes are and are not in contact with target tissue. Imaging techniques, such as fluoroscopy, EAM system (spline proximity to map shell), and intracardiac echocardiography (ICE) cardiac imaging, via such inputs as heart map data 310, can be employed to determine which ablation electrodes are in contact with the tissue. Still other techniques such as a measurement of resistance to fluid flow, via irrigation lumens disposed in each spline, can be employed to determine which ablation electrodes are in contact with target tissue and which ablation electrodes are not in contact with target tissue.

The controller can be configured to apply a pulsed electrical signal to only the selected subset of ablation electrodes (i.e., some or all of the electrodes identified as being in contact with tissue) and not to the remainder of the ablation electrodes. In one example, the pulsed signal to effect ablation is applied in a monopolar mode, and the controller can be configured to apply the pulsed electrical signal to one or more electrodes in contact with the target tissue. In one example of the pulse electrical signal to effect ablation in bipolar mode, a minimum of two ablation electrodes are activated to generate the electrical field. The remainder of the electrodes, i.e., the electrodes not in contact with the tissue, are not used to generate electrical fields for ablation.

In some embodiments, the configuration of the ablation electrodes in contact with the target tissue is used to select characteristics of the pulsed electrical signal, such as the strength, duration, waveform, and other characteristics, is selected based on the configuration. The characteristics of the pulsed electrical signal applied to the ablation electrodes in contact with the target tissue can be based on an output from a lookup table. The remaining electrodes, or electrodes not in contact with the target tissue are not activated. In one example, the output can be based on features such as the ablation electrodes in contact with the tissue, the spacing of the ablation electrodes in the electrode array, whether the ablation is configured in a monopolar mode or a bipolar mode, and other information determined with the pre-pulse electrical signal or determination of which electrodes are in contact, such as impedance or current measurements, which can provide information on the robustness of contact. The waveform applied to the ablation electrodes in contact with the target tissue can be provided as a single preset or adapted into firing pairs. The ablation electrodes in contact with the target tissue can be determined between each application of the pulsed signal. Further, impedance or current measurements can be taken at the ablation electrodes in contact with the target tissue to determine the pulsed electrical signal to apply to the ablation electrodes in contact with the target tissue.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system to perform electroporation ablation of target tissue in a chamber of a patient's heart, the system comprising:
a catheter including an electrode assembly having a plurality of ablation electrodes, wherein the catheter is adapted to position the electrode assembly in contact with the target tissue; and
a controller configured to:
determine a first subset of ablation electrodes in contact with tissue and a second subset of ablation electrodes that are not in contact with the target tissue; and
selectively apply a pulsed electrical signal to only the first subset of ablation electrodes.

2. The system of claim 1, wherein the controller is configured to generate and apply a pre-pulse electrical signal to the plurality of ablation electrodes to determine the first subset of ablation electrodes and the second subset of ablation electrodes.

3. The system of claim 2, wherein the pre-pulse electrical signal is one of a continuous signal and a selectively applied, discrete pre-pulse signal.

4. The system of claim 1, wherein the controller is configured to apply a first pre-pulse signal to the plurality of ablation electrodes with the catheter not in contact with the target tissue and a second pre-pulse signal to the plurality of ablation electrodes with the catheter in contact with the target tissue.

5. The system of claim 4, wherein the controller is configured to apply the first pre-pulse signal with the electrode assembly disposed in a blood pool.

6. The system of claim 2, wherein the electrode assembly further includes a plurality of sense electrodes.

7. The system of claim 6, wherein the controller is further configured to generate and apply the pre-pulse electrical signal to the plurality of sense electrodes in addition to the plurality of ablation electrodes to determine the first subset of ablation electrodes and the second subset of ablation electrodes.

8. The system of claim 2, wherein the controller is configured to measure a target tissue impedance responsive to the application of the pre-pulse signal to the plurality of ablation electrodes.

9. The system of claim 1, wherein the controller is configured to select one or more characteristics of the pulsed electrical signal based on a configuration of the first subset of ablation electrodes.

10. The system of claim 9, wherein the one or more characteristics of the pulsed electrical signal includes one or more waveform parameters.

11. A system to perform electroporation ablation of target tissue in a chamber of a patient's heart, the system comprising:
a catheter including an electrode assembly having a plurality of ablation electrodes, wherein the catheter is adapted to position the electrode assembly in contact with the target tissue; and
a controller configured to:
calibrate the plurality of ablation electrodes when the plurality of ablation electrodes are at a first location not in contact with the target tissue;
determine, with the plurality of ablation electrodes at a second location, a subset of the plurality of ablation electrodes that are in contact with the target tissue based on the calibration; and
selectively apply a pulsed electrical signal only to the subset of ablation electrodes.

12. The system of claim 11, wherein the controller is configured to apply a first pre-pulse signal to the plurality of ablation electrodes at the first location to calibrate the plurality of ablation electrodes, and to apply a second pre-pulse signal to the plurality of ablation electrodes at the second location to determine the subset of ablation electrodes in contact with the target tissue.

13. A method for use with a catheter including an electrode assembly having a plurality of ablation electrodes, wherein the catheter is adapted to position the electrode assembly in contact with the target tissue, the method comprising:
determining a subset of the plurality of ablation electrodes that are in contact with the target tissue; and
selectively applying a pulsed electrical signal only to the subset of ablation electrodes.

14. The method of claim 13, further comprising selecting one or more characteristics of the pulsed electrical signal based on a configuration of the subset of ablation electrodes.

15. The method of claim 13, wherein determining the subset of ablation electrodes in contact with the target tissues includes applying a pre-pulse electrical signal to the plurality of ablation electrodes.

16. The method of claim 15, further comprising:
positioning the electrode assembly at a first location not in contact with the target tissue and applying the pre-pulse signal to the plurality of ablation electrodes at the first location;
establishing a first signal response measurement responsive to applying the pre-pulse signal to the plurality of ablation electrodes at the first location;
positioning the electrode assembly at a second location and applying the pre-pulse signal to the plurality of ablation electrodes at the second location; and
determining a second signal response measurement responsive to applying the pre-pulse signal to the plurality of ablation electrodes at the second location,
wherein determining the subset of ablation electrodes in contact with the target tissue is based on the first signal response measurement and the second signal response measurement.

17. The method of claim 16, wherein the first signal response measurement and the second signal response measurement each comprises an impedance responsive to the pre-pulse signal.

18. The method of claim 15, wherein the pre-pulse signal is a continuous signal or a selectively applied, discrete pre-pulse signal.

19. The method of claim 15, wherein the applying the pre-pulse electrical signal to the plurality of ablation electrodes includes applying the pre-pulse signal to the plurality of ablation electrodes with two or more of the plurality of electrodes configured in a bipolar mode.

20. The method of claim 19, wherein the applying the pulsed electrical signal to the plurality of ablation electrodes includes applying the pulsed electrical signal to one or more of the plurality of ablation electrodes configured in a monopolar mode in combination with an indifferent electrode.
